(19) United States
(12) Patent Application Publication
(10) Pub. No.: US 2019/0254581 A1
(43) Pub. Date: Aug. 22, 2019

(54) SYSTEM AND METHOD FOR DIAGNOSING AND ASSESSING THERAPEUTIC EFFICACY OF MENTAL DISORDERS

(71) Applicant: Rutgers, The State University of New Jersey, New Brunswick, NJ (US)

(72) Inventors: Thomas V. Papathomas, Madison, NJ (US); Attila J. Farkas, New Brunswick, NJ (US)

(21) Appl. No.: 16/325,489

(22) PCT Filed: Sep. 13, 2017
(86) PCT No.: PCT/US17/51345

§ 371 Date: Feb. 14, 2019

Related U.S. Application Data
(60) Provisional application No. 62/393,841, filed on Sep. 13, 2016.

(51) Int. Cl. A61B 5/16; A61B 5/00; G16H 50/20; G16H 20/70; G06T 15/04; G06T 11/00; G06T 11/60; G06T 13/40

(52) U.S. Cl. CPC A61B 5/163; A61B 5/4848; A61B 5/742; G16H 50/20; G06T 2210/41; G06T 15/04; G06T 11/001; G06T 11/60; G06T 13/40; G16H 20/70

(57) ABSTRACT

Systems and methods for generating and rendering one or more images, such as in an animated image sequence, of the virtual multi-dimensional object on a display screen for testing a person's susceptibility to a Depth Inversion Illusion (“DII”). The methods also include collecting first information indicating the person's perceptual response to the DII; adjusting a strength of the DII by manipulating a texture that is mapped onto the virtual multi-dimensional object; collecting second information indicating the person's perceptual response to the DII; using the first and second information to determine differences between the person's perceptual responses to the DII and reference perception responses of a group of control subjects to the DII; and analyzing the differences to determine a severity of the person's mental illness or to assess therapeutic efficacy.

### CROSS-REFERENCE TO RELATED APPLICATIONS

This patent document claims priority under 35 U.S.C. § 119(e) to U.S. Provisional Patent Application No. 62/393,841, filed Sep. 13, 2016. This Provisional U.S. application is incorporated herein by reference in its entirety.

### FIELD

This document relates generally to systems and methods for diagnosing and assessing therapeutic efficacy of mental disorders, such as schizophrenia, and in particular to generating and rendering stimuli on a display screen for testing a person's susceptibility to a Depth Inversion Illusion (“DII”).

### BACKGROUND

Early detection and therapy of mental disorders or mental illness such as schizophrenic psychoses has become a widely accepted goal in psychiatry. Centers for early detection and intervention have been set up worldwide. For example, the UK Government has decided to systematically invest in early detection and intervention as “the rationale for early intervention is overwhelming.”

Whereas until some time ago early diagnosis and intervention in schizophrenia concentrated on clear-cut, frank schizophrenia, during the last years some centers have also started to treat patients even before a clear diagnosis could be established. The rationale behind this is that these disorders often begin many years before first clear symptoms occur with quite unspecific changes and prodromal symptoms and/or very brief, transient or mild ‘attenuated’ (subthreshold) psychotic symptoms, but often have deleterious consequences already in these early stages. However, reliable methods for an early detection already in this phase of beginning schizophrenia do not yet exist.

### SUMMARY

The present disclosure concerns systems and methods for diagnosing and assessing therapeutic efficacy of a mental illness. The methods comprise: generating one or more images of a virtual multi-dimensional object (e.g., a hollow mask of a face), where the one or more images can be rendered in an animation sequence; rendering the one or more images on a display screen of a computing device (e.g., a portable computing device such as a smart phone) for testing a person's susceptibility to a Depth Inversion Illusion (“DII”); collecting information indicating the person's perceptual responses to the DII in a series of trials in which the strength of the DII is varied by manipulating a texture that is mapped onto the virtual multi-dimensional object; using the collected information to determine differences between the person's perceptual responses to the DII and reference perception responses of a group of control subjects to the DII; and analyzing the differences to determine a severity of the person's mental illness or to assess therapeutic efficacy.

In some scenarios, the collected information comprises sensor data specifying tracked eye movements and/or user-input information specifying a person's answer to at least one question.

In those or other scenarios, the system includes adjusting the strength of the DII in different trials by adding or removing a random noise texture from the virtual multi-dimensional object.

In those or yet other scenarios, the system determines the differences by: plotting the data points on a graph having a two-dimensional coordinate system, the first set of data points representing the person's perceptual responses to the DII at different strength levels; and respectively comparing the first set of data points to second set of data points representing perception responses of a group of control subjects to the DII at the different strength levels. An x-axis of the two-dimensional coordinate system lists stimuli that were used during the method. A y-axis of the two-dimensional coordinate system comprises values specifying the strength of the DII for corresponding stimuli.

In some scenarios, the system generates one or more images of a virtual multi-dimensional object (e.g., a hollow mask of a face) and render the one or more images on a display screen of a computing device for testing a person's susceptibility to a Depth Inversion Illusion (“DII”). The system may render the one or more images in an animation sequence.

In generating the one or more images of the virtual multi-dimensional object, the system may use texture titration to generate a composite image based on a face texture image and the DII strength level. The system may also apply planar texture projection to map the composite image onto virtual multi-dimensional object to generate a mapped 3-D model, and generate the image of the virtual multi-dimensional object based on a view of the mapped 3-D model from a viewing angle.

In generating the composite image, the system may generate a composite dot texture image by: generating a dot texture image comprising a plurality of binary cells each comprising a plurality of pixels, each cell being defined randomly by a value of white or black with equal probability; generating one or more scaled dot texture images based on the dot texture image, wherein each scale dot texture image is scaled down a percentage from the dot texture image; aligning the one or more scaled dot texture images with the dot texture image; and overlaying the one or more aligned dot texture images to the dot texture image to generate the composite dot texture image. Each pixel in the composite dot texture image has a value of black if at least one corresponding pixel in the dot texture or the one or more aligned dot texture images has a value of black; otherwise the pixel in the composite dot texture image has a value of white.

In generating the composite image, the system may further perform the steps of: aligning the composite dot texture image with the face texture image; and overlaying a first proportion of the aligned composite dot texture image to a second proportion of the face texture image, wherein the first and second proportions are summed at a value of one. The system may change the values of the proportions based on the DII strength level.

### DESCRIPTION OF THE DRAWINGS

The present solution will be described with reference to the following drawing figures, in which like numerals represent like items throughout the figures.

FIG. 1 is an illustration of an illustrative computing system.

FIG. 2 is a flow diagram of an illustrative method for determining a severity of a person's mental illness and/or assessing therapeutic efficacy.

FIG. 3 shows an illustrative map comprising a graph with data points plotted thereon.

FIG. 4 shows an example diagram of a process for generating an image of a virtual multi-dimensional object.

FIG. 5A shows an example diagram of a process for generating a composite image.

FIG. 5B shows an example diagram of a process for generating a composite dot texture image.

FIG. 6A shows an example of a face texture image.

FIG. 6B shows an example of a composite dot texture image.

FIG. 6C shows an example of a composite image.

FIG. 7A shows an example of a mesh of a gender-neutral 3-D mask model of a virtual multi-dimensional object.

FIG. 7B shows an example of an image of the virtual multi-dimensional object in FIG. 7A via the steps described in FIG. 4.

FIGS. 8A-8C illustrate examples of images of the virtual multi-dimensional object in various Depth Inversion Illusion (“DII”) strength levels.

FIGS. 9A-9D illustrates examples of random-dot texture images at various scales.

FIG. 10A illustrates an example of a layering process for aligning the random-dot texture images in FIGS. 9A-9D.

FIG. 10B illustrates an example of a composite dot texture image as a result of the process in FIG. 10A.

### DETAILED DESCRIPTION

It will be readily understood that the components of the present solution as generally described herein and illustrated in the appended figures could be arranged and designed in a wide variety of different configurations. Thus, the following more detailed description of the present solution, as represented in the figures, is not intended to limit the scope of the present disclosure, but is merely representative of various implementations of the present solution. While the various aspects of the present solution are presented in drawings, the drawings are not necessarily drawn to scale unless specifically indicated.

The present solution may be embodied in other specific forms without departing from its spirit or essential characteristics. The described embodiments are to be considered in all respects only as illustrative and not restrictive. The scope of the present solution is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present solution should be or are in any single embodiment of the present solution. Rather, language referring to the features and advantages is understood to mean that a specific feature, advantage, or characteristic described in connection with an embodiment is included in at least one embodiment of the present solution. Thus, discussions of the features and advantages, and similar language, throughout the specification may, but do not necessarily, refer to the same embodiment.

Furthermore, the described features, advantages and characteristics of the present solution may be combined in any suitable manner in one or more embodiments. One skilled in the relevant art will recognize, in light of the description herein, that the present solution may be practiced without one or more of the specific features or advantages of a particular embodiment. In other instances, additional features and advantages may be recognized in certain embodiments that may not be present in all embodiments of the present solution.

Reference throughout this specification to “one embodiment”, “an embodiment”, or similar language means that a particular feature, structure, or characteristic described in connection with the indicated embodiment is included in at least one embodiment of the present solution. Thus, the phrases “in one embodiment”, “in an embodiment”, and similar language throughout this specification may, but do not necessarily, all refer to the same embodiment.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”. In this disclosure, the reader should understand that, while the term schizophrenia generally refers to a specific class of diagnosis a variety of mental illnesses or disorders, herein the term should be understood to mean schizophrenia or any of a variety of mental conditions which may alter an individual's ability to perceive certain visual experiences such as DII.

The present disclosure concerns implementing systems and methods for diagnosing and assessing therapeutic efficacy of schizophrenia. The methods may be implemented in hardware, such as in a computing system or a mobile device. The methods may be implemented in software, such as a software application. The software application is a diagnostic software system that may be hosted by a variety of portable platforms (e.g., tablet, laptop, desktop computer, smart device, etc.). The software utilizes animations of computer-generated three-dimensional (3-D) objects. The basic stimulus is a 3-D hollow mask object used for testing a patient's susceptibility to the strength level of depth inversion illusion (DII). DII represents an illusion of visual perception, which inverts the perception of a hollow object, e.g. a hollow face into a normal face. The hollow 3-D mask creates the Hollow-Mask Illusion (“HMI”), which is a member of the class of Depth Inversion Illusions (“DII”) by being perceived as a regular convex mask. Schizophrenia patients are less sensitive to DII induced by a hollow mask. The software design makes it possible to increase and decrease the strength of the DII by manipulating the texture that is mapped onto the hollow mask object. This method establishes a sensitive diagnostic procedure that maps the differences in perceptual responses of patients and healthy controls. The analysis of differences of the perceptual responses may be used as an indicator of disease severity and also has the potential to assess therapeutic efficacy.

The software provides a new diagnostic tool that allows clinicians to use mobile or portable electronic devices to bring the test to the patient, rather than bring the patient to the clinic, with obvious advantages for reliability in timely examination. The present solution may be applied both for diagnostic purposes and for assessing the efficacy of therapeutic regiments for schizophrenia patients. Early detection and treatment significantly improves patients' response to treatment and could prevent a progression to full relapse by prompting adequate clinical intervention. The proposed solution is deliberately designed to be time effective and has the potential to readily provide assessment reports.

To reduce the increasing cost and to prevent full relapses, the rapid detection and diagnosis of the illness might be crucial. There are many variables that hinder rapid detection. Patients with mental illness may be unreliable. The patients are frequently late or not showing up on the scheduled time slot. The cost and logistics of transportation to and from the laboratory poses additional problems in the process. The present solution makes it possible for portable devices (e.g., laptops, tablets, smart devices, etc.) to host the entire diagnostic procedure.

The present solution provides a diagnostic tool that is portable and uses computer graphics and animation to assess disease severity or therapeutic efficacy of mental illnesses. The proposed application has the advantage to connect to an online database and readily compare the patient's response to baseline responses of healthy controls. The proposed solution has the ability to directly send results to other clinicians who can perform a wide variety of analysis at the same time. This factor also has the potential to fill a yet unmet market need in healthcare.

Early Detection and Treatment of Schizophrenia

When dealing with mental illnesses such as schizophrenia, a main question is whether and at what stage early intervention such as treatment with low-dose atypical neuroleptics is indicated. This question confronts researchers and clinicians with the ethical dilemma between diagnosing/treating this disorder either too late or too early. On the one hand, the disease process can be very devastating already in the early prodromal stages. On the other hand, it is important not to diagnose/treat too early, because of the potential identification of ‘false positives’, the stigma associated with the diagnosis, the potential side-effects of treatment.

The following discussion attempts to answer the following questions.

Is there really a sound rationale for the early detection and treatment of schizophrenic psychosis?
What are the problems of early detection and treatment?
How could one improve early detection?

Material and Methods

A selective review of recent literature was performed to answer these key questions. Medline and PsycINFO (2000-2004) were searched using mainly combinations of the key words: schizophrenia; first episode; (high) risk; early diagnosis; risk factors; and prevention. In addition, previous reviews and books on the topics were used.

Results

Rationale for Early Detection and Treatment of Schizophrenia

The rationale for early detection of schizophrenia is based on several observations: diagnosis and treatment of schizophrenia are often seriously delayed; consequences of the disease are very severe already in the early preclinical, undiagnosed phase of the disorder; and early treatment seems to improve the course of the disease. Each of these observations will be discussed separately below.

The diagnosis and treatment of schizophrenia are often seriously delayed.

i) Duration of Untreated Psychosis (“DUP”): Patients suffer from productive psychotic symptoms, such as delusions or hallucinations, for an average of 1-3 years before this disorder is diagnosed and treated for the first time.
ii) Duration of Untreated Illness (“DUI”): Even before that, patients suffer from an ‘unspecific prodromal phase’ for an average of 2-5 years.

One of the first studies which could show this delay on a methodologically sound basis was an ABC study. In this study, retrospectively the following was shown: the initial signs on average became apparent approximately 4.6 years prior to first admission and diagnosis. First psychotic symptoms occurred on average 2.1 years before first admission.

Consequences of the disease are very severe already in the early preclinical, undiagnosed phase of the disorder. One of the further major findings of the ABC study was that before first admission most patients already suffered from serious impairments and losses in various social domains such as education, work, partnership or independent living. Especially as the disease often strikes individuals when they are still very young and in the midst of their developmental years, consequences for the different social roles are often deleterious. Thus, quality of life is seriously impaired already at first admission and associated with DUP.

Early treatment seems to improve the course of the disease. There is a large body of evidence that early treatment of psychosis can substantially improve treatment response, course and outcome of the disease. Thus, the majority of studies found a statistically significant correlation between long DUP and poor outcome. This is especially true for short-term outcome, but also applies to long-term outcome. While some authors questioned a direct causal link between DUP and outcome, several studies demonstrated that DUP consistently predicted outcome independently of other variables, and that it was not simply a proxy for other factors.

The mechanisms by which DUP influences outcome could be multifold. Thus, ongoing psychosis could have direct ‘neurotoxic’ effects including molecular sensitization and neurodegeneration with symptomatic progression and cognitive deterioration, although there are also studies questioning these theories.

A delay of treatment on the contrary can have very severe consequences. Thus, it has been noted that a longer DUP was associated with an incomplete remission of symptoms, with a worse long-term prognosis, a higher overall dosage of neuroleptics, a worse compliance, higher burden for the family and higher expressed emotion level, a higher rate of re-hospitalizations and higher overall treatment costs. An enhanced risk of depression, suicide and substance abuse is expected if there is a long phase of untreated disease.

It can therefore be stated quite safely that patients should be diagnosed and treated as early as possible. The question, however, is: how early?

Problems of Early Detection and Treatment

Early detection of schizophrenia? An early diagnosis of ‘schizophrenia’ before the diagnostic criteria are fulfilled, might be possible retrospectively, but is ‘per definition’ not possible prospectively.

Early detection of psychosis? Researchers and clinicians have, therefore, concentrated on the early diagnosis of ‘psychosis’ using well-defined criteria for psychotic breakdown. Early treatment of patients who fulfill these criteria aims at reducing the DUP. It seems quite clear that early treatment should start at least as soon as frank psychosis has occurred, as this can substantially ameliorate symptoms and shorten psychotic episodes and thereby avoid or at least ameliorate the immediate negative psychological and social consequences.

Early detection of ‘beginning illness’? Early detection of ‘at-risk status’? However, a reliable detection of the disorder even before frank psychotic breakdown is still not possible prospectively. At this stage of a presumed illness, diagnosis of a disorder (schizophrenia) or a syndrome (psychosis) was not possible. And there is not even enough evidence for a reliable detection of an ‘at-risk status’, let alone a prodromal phase of the disease.

Treatment of such individuals, thus, raises many questions which have not been sufficiently answered as yet, especially ethical ones. Thus, exclusion of the identification and treatment of ‘false positives’ was not possible. These individuals would have to cope with the information on their risk which might be reasonable and comparable to other risk assessments and patient education in medicine. Nevertheless, one must be aware of the special stigma associated with schizophrenia and—as a consequence of this—the special stress put on the individuals confronted with this presumed risk. And, more importantly, those individuals were exposed to potential risks and side-effects of therapy and medication. Nevertheless, during the last years some centers have started treatment in this unspecific prodromal phase, aiming not any more at reducing DUP as has been tried so far, but at reducing DUI.

In some people's opinion, this might be still too early. The prerequisite for such a very early ‘diagnosis’ and intervention would be a more reliable assessment of the at-risk status and also of the early stages of the beginning disease. That means the decision for such very early treatment should be based on more and better empirical evidence. This clearly needs more research.

But what possibilities for enhancing the reliability of such a very early ‘diagnosis’ exist? What should research aim at?

Improvement of Early Detection: Possible Approaches

Early identification of individuals at risk and detection of the very early phases of the disease could theoretically be improved by: (i) identifying more reliable risk factors and indicators of a beginning disease; (ii) using different levels of investigation; and (ii) combining these different risk factors/indicators for a comprehensive ‘multidomain risk assessment’.

What domains are these? What predictors for developing schizophrenia are known? To answer this, a comprehensive search of the literature was performed with a special focus on patients who had been investigated before full-blown schizophrenia had occurred. Retrospectively, such patients are described in first-episode studies, prospectively in genetic high-risk studies and birth-cohort studies. Cross-sectional data was also considered of first-episode patients hypothesizing that the abnormalities they show in different domains such as neuropsychology or neuroradiology might already start before the first psychotic episode. Based on these results, a finding was found that early identification of a beginning disease or individuals at risk should theoretically be possible in several domains, mainly the following: (i) early risk factors for schizophrenia (genetic risk, obstetric complications, etc.); (ii) psychopathology; (iii) other indicators of beginning disease (social decline, help seeking behavior, etc.); (iv) neuropsychology; (v) neurophysiology; and (vi) neuroimaging.

In the following, the results of the review will be briefly summarized with an emphasis on new findings from the last years.

Early risk factors for schizophrenia. Apart from the well-known genetic risk, other early risk factors such as obstetric complications or developmental and behavioral problems in childhood have been described. High-risk studies, birth-cohort studies and retrospective and follow-back studies report that future schizophrenic patients have delayed developmental milestones, speech and behavioral difficulties and lower IQ scores than non-cases. Recent publications have confirmed earlier studies. Thus, for example, an analysis of a large birth-cohort found that the ages at learning to stand, walk or become potty-trained each related to subsequent incidence of schizophrenia and other psychoses. Also, in a birth-cohort study, significant impairments were found in neuro-motor and cognitive development as well as that of receptive language. Furthermore, emotional problems and interpersonal difficulties were found among children later diagnosed as having schizophreniform disorder. In offspring of schizophrenic patients, the following has been found: childhood deficits in verbal memory, gross motor skills and attention to predict schizophrenia-related psychoses in adulthood. The following factors in childhood and adolescence have been found to predict schizophrenia: problems in motor and neurological development, deficits in attention, poor social competence, positive formal thought disorder-like symptoms and severe instability of early rearing environment.

Psychopathology. Studies have also confirmed the importance of early psychopathological abnormalities and so-called prodromal symptoms. Children of schizophrenic patients were followed into adulthood within the New York High Risk Project. They rated video tapes of these children and found thought disorder as well as negative symptoms in those children who went on to develop schizophrenia.

The predictive value of prodromal symptoms has been investigates. The Bonn Scale for the Assessment of Basic Symptoms was used to predict schizophrenic disorder in a sample of 385 patients. After a mean period of 9.6 years, 79 (49.4%) of 160 patients, who could be re-examined, had in fact developed schizophrenia. The original presence of prodromal symptoms predicted schizophrenia with a probability of 70% (specificity 0.59, false positive predictions 20%).

A prospective examination of the predictive power of certain mental state and illness variables was performed. They included symptomatic individuals with either a family history of psychotic disorder, schizotypal personality disorder, subthreshold psychotic symptoms or brief transient psychotic symptoms. Of a total sample of 49, 40.8% developed a psychotic disorder within 12 months. Highly significant predictors of transition to psychosis were: long duration of prodromal symptoms; poor functioning at intake; low-grade psychotic symptoms; depression; and disorganization. Combining some predictive variables yielded a strategy for psychosis prediction with good sensitivity (86%), specificity (91%), positive predictive value (80%) and negative predictive value (94%). These results, the authors state ‘lay the groundwork for the development of targeted intervention or indicated prevention models’. The results of an even larger sample of 104 ‘ultra-high-risk’ young people was published. Again, these results showed a specificity of 93%, but only a moderate sensitivity of 60%.

Other indicators of the disease. In addition to psychopathology, other indicators of beginning schizophrenia such as changes of social behavior or deterioration in the fulfillment of social roles have also been identified as important. The importance of a decline of social functioning for predicting psychotic breakdown has been confirmed by scientists.

Neuropsychological and motor deficits. Recent studies confirmed findings about neuroleptic-free first episode schizophrenic patients and individuals at risk having generalized neuropsychological deficits, especially concerning (sustained) attention, abstraction, (verbal) learning, (verbal) memory and executive function.

Regarding individuals at risk, a report was published on 157 individuals at risk (at least two family members with schizophrenia) from the Edinburgh High Risk Study. When compared with 34 controls and the general population, these 152 individuals showed a poorer performance on tests of intellectual function, especially verbal IQ, executive function and memory. This suggests that what is inherited is not the disorder itself but a state of vulnerability manifested by neuropsychological impairment, which although subtle, could distinguish those at risk from control subjects. Scientists have showed attention deficits in siblings of schizophrenia patients, if index patients suffered from severe attention deficits themselves. In the Basel FePsy (Früherkenung von Psychosen) study, 32 individuals at risk for schizophrenia were compared with 32 healthy controls and found impairments in different neuropsychological test parameters, mainly with prolonged reaction times in individuals at risk.

Also neurological abnormalities, such as dyskinesias, Parkinsonian signs and neurological soft signs have been found in neuroleptic-naïve schizophrenia patients. It has been reported that first episode patients show an excess of neurological soft signs especially in the areas of motor coordination and sequencing, sensory integration and developmental reflexes. Correlations between the soft signs and cognitive functions have been shown.

In individuals at risk, delayed motor development, poor motor skills and also increased rates of neurological soft signs have been described. It has therefore been suggested that motor abnormalities may constitute markers of vulnerability. A significant amount of ‘sensory integration abnormalities’ has been detected in individuals at risk (at least two close relatives with schizophrenia) compared with healthy controls. In one study, individuals at risk showed a significant impairment of dexterity and of arm/hand and wrist/finger velocity.

Previous studies also documented deficiencies in eye movements in individuals at risk and patients with first episode schizophrenia. Individuals at risk (identified by the Chapman Psychosis-Proneness Scale) have been found to have more aberrant smooth pursuit eye tracking than controls. In one study, an increased number of correction saccades in smooth pursuit eye movements was found. Also in relatives of patients with schizophrenia, deficits of the saccadic system and eye tracking dysfunction were detected.

Neurophysiology. Electro-EncephaloGraphy (“EEG”) is on the one hand used to exclude organic psychosis, on the other hand to identify EEG-characteristics in schizophrenia. In a review, an analysis of 65 studies of individuals with schizophrenia was performed. This analysis found that the percentage of abnormal EEGs in never medicated patients with schizophrenia ranged between 23% and 44%, in healthy controls between 7% and 20%. Especially quantitative EEG may be of value in a multi-domain approach when correlated with other parameters such as psychopathology or magnetic resonance imaging.

Magnetic resonance imaging. Manifold structural changes of the brain have also been described in first episode schizophrenia and in individuals at risk. In a very important study, 75 individuals at risk were scanned. 23 of whom developed psychosis within 12 months. Those who developed psychosis had already at baseline shown less grey matter in certain brain areas when compared with those who did not develop psychosis. Furthermore, those with progression to frank psychosis also showed progressive grey matter reduction within 12 months.

Multi-domain approach. Some projects now combine different assessment methods, respectively domains of investigation. Thus, not only psychopathology but also neuroradiology has been found to be relevant for the prediction of transition to psychosis. In a sample of 49 individuals at risk, the best predictors were: duration of symptoms longer than 100 days; global assessment of functioning score<51; Brief Psychiatric Rating Scale (“BPRS”) total score>15; BPRS psychotic subscale>2; Scale for the Assessment of Negative Symptoms (“SANS”) attention score>1; Hamilton depression score>18; cannabis dependency; high maternal age at birth; and a normal left hippocampus size (in contrast to the group without progression to psychosis which had shown reduced hippocampal volumes).

These recent findings confirm that very early detection can in fact become more reliable, if in addition to clinical prodromal symptoms or other risk factors and early indicators of vulnerability and/or beginning psychosis are taken into account.

### Discussion

Early detection and treatment of schizophrenia is important and possible. It should in future not only concentrate on the early detection of schizophrenia and frank psychosis, but also on the identification of individuals at risk and especially on that subgroup of at-risk individuals who already show signs of a beginning disease. In these individuals a reliable prediction of psychotic breakdown should be a major goal. As first studies have shown this might be possible, but the empirical basis for this still has to be improved.

Early detection clinics would for the moment thus have the following aims.

i) First, early detection and treatment of clear schizophrenia and frank psychosis to reduce DUP. It has been shown that this is possible through early detection programs. Therefore, ‘the prime focus for the moment should be on the recognition and phase-specific management of patients from the point they cross the boundary to a frank psychotic illness’.
ii) Second, differential diagnosis. Thus, for example, in an Early Detection Clinic (“EDC”), a wide range of organic reasons for the presented psychopathology (such as epilepsy, encephalitis and even chronic subdural hemorrhage) were detected.
iii) Third, early detection clinics should also contribute to a more reliable assessment of the risk for schizophrenia in individuals suffering from still unclear clinical conditions and suspected beginning schizophrenia. In these individuals, it was shown, however, not to talk about early ‘diagnosis’ but rather about early ‘risk assessment’. Ethically, in these patients specific neuroleptic treatment usually is not yet justified, as the criteria for intervention are not clear enough until now. For the moment, these individuals should be very cautiously informed about their potential risk, should be cared for and receive unspecific treatment, if they suffer from unspecific symptoms, which some of them already do to quite some extent. Additionally, they should carefully be observed so that in case of transition to psychosis specific treatment can be implemented immediately.

Research at the same time has to make further efforts towards improving the empirical basis for early detection and treatment of schizophrenic psychoses and thereby towards solving the current ethical dilemma of neither diagnosing and treating too late nor too early. Research in this field thus is an ‘ethical obligation’. The great hope is that individuals suffering from so far unexplained symptoms could, in the future, be more clearly informed about their possible risk for developing schizophrenia and counselled concerning preventive measures. Treatment could then be targeted not only on actual symptoms, but also—in a more specific way—aim at preventing psychotic breakdown in the sense of an ‘indicated prevention’. Ideally treatment would be started stepwise according to the intensity and profile of the risk, and would in different levels of intervention sequentially use different therapeutic strategies such as supportive measures, psychotherapy and/or low-dose neuroleptics—based on empirical evidence.

Relapse In Schizophrenia

Symptomatic relapse in schizophrenia is both distressing and costly. It can devastate the lives not only of patients, but also of their families. The debilitating symptoms require specialist health care interventions and targeted treatments, with potentially high costs. It has been estimated, for example, that relapse cost $2 billion just for readmissions to hospital in the United States of America, almost a decade ago. There is no equivalent estimate for the United Kingdom. This study aimed to compare costs, clinical outcomes and Quality of Life (“QoL”) for patients with schizophrenia in the United Kingdom according to whether or not they had experienced a relapse in the previous 6 months.

Method

Study Sample

Patients were randomly selected from current (active) psychiatric case-loads drawn from urban and suburban areas of the English city of Leicester. Consultant psychiatrists or senior responsible medical staff were approached by a project research psychiatrist and asked for a list of patients with a possible diagnosis of schizophrenia. Full lists were obtained from five consultants covering city and suburban catchment areas of Leicester. An additional five consultants were also approached to identify patients with the diagnosis who had experienced a relapse within the past 6 months. Patients were excluded if they were living outside this area when the sampling was undertaken. Patients from rural areas of Leicestershire were excluded. The sampling procedure was designed to recruit equal numbers of relapse and non-relapse cases.

Patients were included as participants if they had received a diagnosis of schizophrenia according to DSM-IV criteria (American Psychiatric Association, 1994), had no other psychosis, were aged 18-64 years, and gave their informed consent. Patients were excluded from the study if they were roofless, continuously hospitalized for 12 months or more, about to move residence, already participating in a clinical trial, or unable to participate for language reasons. Although such biases were not specifically controlled for, clinicians took every step to avoid biases in the socio-economic and demographic profiles of patients.

Relapse Criteria

Many alternative definitions of relapse in schizophrenia have been published. These include number of admissions to hospital, detention under a section of the Mental Health Act (“MHA”), attendance at an acute day care center, change of antipsychotic agent, increased staff input and/or more intensive care staff management, and a significant change in accommodation. Relapse was identified retrospectively in this study as the re-emergence or aggravation of psychotic symptoms for at least 7 days during the 6 months prior to the study. In addition to instances of relapse pointed out by clinical staff, recorded changes in mental state were regarded as significant and amounting to relapse if there was a clearly documented assessment of a relapse. A change in management as appropriate might also have occurred but not necessarily, and not all relapses led to readmission. Relapse could thus be identified in cases of patients who had been admitted to hospital in the past 6 months, who had consulted their psychiatrist and had had their medication changed for deterioration in their condition, or who had had an increase in intensive support at home from the community mental health team. A planned hospital admission was not classed as a relapse. A research team specialist registrar advised the researcher on any case-note descriptions or accounts from staff that were unclear.

Instrumentation

Data was collected for this study. Data collection was based on information obtained directly from case notes and from interviews with the patients in which rating scales were completed (patients gave informed written consent). The information had not been extracted for any other or prior reason.

The following were used: a Positive and Negative Syndrome Scale (“PANSS”); a question from a Clinical Global Impression scale (“CGI”) covering severity of illness; a Global Assessment of Functioning (“GAF”); a Lehman Quality of Life scale (“LQLS”); a visual analogue scale from the EuroQoL EQ-5D health-related quality of life measure; and a Client Service Receipt Inventory (“CSRI”). Unit costs attached to services were national average figures for the period over which clinical and service use data were collected.

Statistical Analyses

Depending on the distribution of key variables, parametric (independent t-test) and non-parametric tests were carried out to check for significant differences in mean costs, clinical and QoL outcomes by relapse status. The Pearson chi-squared statistic was used to test for significant differences between categorical measures and relapse status, and for other relapse criteria.

The survey design also permitted multivariate analysis to examine simultaneously some of the potential correlates of relapse status and costs, although it should be noted that the study did not include a full range of possible associations with relapse. First, a Generalized Linear Model (“GLM”) with a logit link function was used to predict whether a patient had experienced a relapse or not. The logit GLM is similar to the standard logistic model but also produces a measure of dispersion (the variance of the unexplained part of the model). Odds ratios are presented which show the likelihood of relapse given particular patient characteristics. Second, because costs were skewed to the right (although only 5% were zero values), standard ordinary least squares estimates were inappropriate. The results presented are based on a reduced-form GLM model, with a log link function and a Gaussian variance function. Compared with other standard GLM specifications, this produced the best-fitting model in terms of mean predicted cost levels. It also produced the most efficient estimates in terms of lower standard errors and smaller confidence intervals. The statistical analyses were carried out using the Statistical Package for the Social Sciences version 9 for descriptive comparisons and STATA version 6 for the multivariate analyses.

Results

Sample

257 patients were identified as being potentially eligible to participate in the study. Of these, 12 refused to take part, 67 were not interviewed because of staff concerns, 12 could not be contacted, and 9 were judged by the interviewer to be too ill. In three cases, it was felt to be unsafe to see the patient at home.

A total of 145 patients completed interviews in the study. There were 77 relapse cases and 68 non-relapse cases. Another 9 patients who were also interviewed were excluded because of incomplete records or inconsistent data. The limited information available on them suggests that most would have been assigned to the non-relapse group and, if included, their cases would have had little impact on average costs.

Relapse and Patient Characteristics

Relapse status was defined on the basis of re-emergence or aggravation of psychotic symptoms. TABLE 1 lists other patient characteristics previously employed to define relapse. Not surprisingly, relapse cases were characterized by higher rates of hospitalization (63%), re-emergence of psychotic symptoms (60%) and aggravation of positive or negative symptoms (43%), and an increased level of staff input or more intensive case staff management (33%) (all P<0.05).

TABLE 1
Criteria for assignment to relapse or non-relapse study group

| | | Relapse |
|---|---|---|
| | Non-relapse | (n = 77) |
| Variable | (n = 68) % | % |
| Significant change in management directly | 0 | 100 |
| related to illness or treatment side-effects¹ | | |
| Change in clinical state | | |
| Re-emergence of psychotic symptoms² | 0 | 60 |
| Aggravation of positive or negative symptoms² | 0 | 43 |
| Change in management | | |
| Hospital admission in past 6 months² | 0 | 63 |
| Detention under section of Mental Health Act² | 0 | 20 |
| Acute day care³ | 0 | 5 |
| Change of antipsychotic agent² | 0 | 21 |
| Increased staff input, more intensive | 0 | 33 |
| case staff management² | | |
| Significant change in accommodation³ | 0 | 5 |

¹Chi-squared test not computed.
²Chi-squared test significant at P < 0.05.
³Chi-squared test not significant at P = 0.05.

Compared with the non-relapse group, patients who had recently experienced a relapse had been more recently admitted to a psychiatric ward (using actual years: 1997 and 1992, P<0.05), and experienced a higher number of admissions (5.6 and 3.3, P<0.05). Although patients in the non-relapse group appeared to have spent longer in hospital, the difference was not significant as shown in TABLE 2. There was no difference between the relapse and non-relapse groups with respect to gender, ethnic group, marital status, employment status or highest level of education, as shown in TABLE 3. Relapse patients were more likely to be living alone (P<0.05). Mean ages were 37.9 (s.d.=10.7) years for relapse patients and 41.1 (s.d.=11.1) years for non-relapse patients (not significantly different).

TABLE 2
Characteristics of service contact prior to study entry

| | Non-relapse | |
|---|---|---|
| | (n = 68) | Relapse (n = 77) |
| Variable | mean (s.d.) | mean (s.d.) |
| Year of first contact with mental | 1985 (8.7) | 1987 (8.3) |
| health services because of | | |
| psychotic illness¹ | | |
| Year first admitted to psychiatric ward² | 1986 (8.7) | 1989 (7.7) |
| Year of most recent admission to | 1992 (7.0) | 1997 (3.9) |
| psychiatric ward² | | |
| Number of times admitted to | 3.3 (4.1) | 5.6 (4.8) |
| psychiatric ward² | | |
| Longest admission to psychiatric | 7.1 (29.6) | 4.6 (2.8) |
| ward (months)¹ | | |

¹Independent t-test not significant at P = 0.05.
²Significant at P < 0.05 (similar results achieved using non-parametric tests).

TABLE 3
Socio-economic and demographic
characteristics of the participants

| | Non-relapse | Relapse |
|---|---|---|
| Variable | (n = 68) % | (n = 77) % |
| Gender | | |
| Female | 47.1 | 32.8 |
| Ethnic group¹ | | |
| White | 82.4 | 83.1 |
| Black Caribbean | 4.4 | 2.6 |
| Indian | 11.8 | 13.0 |
| Other | 1.4 | 1.3 |
| Marital status¹ | | |
| Single | 55.9 | 74.0 |
| Married/cohabiting | 26.5 | 11.7 |
| Divorced/separated | 16.2 | 10.4 |
| Widowed | 1.4 | 3.9 |
| Highest educational level¹ | | |
| Primary | 4.4 | 1.3 |
| Secondary | 88.2 | 76.6 |
| Tertiary/further | 4.4 | 13.0 |
| Other (not specified) | 2.9 | 9.1 |
| Living arrangements² | | |
| Alone at home | 19.1 | 37.7 |
| With family/others | 53.0 | 35.1 |
| Collective | 22.1 | 11.7 |
| accommodation | | |
| Other (not specified) | 5.8 | 15.6 |
| Employment¹ | | |
| Not working | 94.1 | 97.4 |

¹Pearson χ² not significant a P = 0.05.
²Significant at P < 0.05.

Clinical Health and Quality of Life

Although higher scores on the PANSS and the CGI suggested worse symptoms for relapse compared with non-relapse cases, the differences were not statistically significant. However, GAF scores indicated worse symptoms for relapse patients (P<0.05; TABLE 4).

TABLE 4
Clinical characteristics and quality of life

| | Non-relapse | |
|---|---|---|
| Clinical and QoL scales | (n = 68) % | Relapse (n = 77) % |
| PANSS | | |
| Positive scale¹ | 12.9 | 15.4 |
| Negative scale¹ | 15.0 | 15.8 |
| General psychopathology¹ | 31.0 | 32.1 |
| CGI¹ | 3.5 | 4.6 |
| GAF² | 57.8 | 52.6 |
| Lehman QoL | | |
| General life satisfaction (D-T scale)¹ | 4.3 | 3.8 |
| Living arrangements (D-T scale)² | 15.0 | 13.3 |
| Daily activities (score)¹ | 4.1 | 3.8 |
| Functioning (D-T scale)¹ | 2.7 | 2.8 |
| Family | | |
| Talk/get together (score)¹ | 7.5 | 7.2 |
| Relationship (D-T scale)¹ | 9.6 | 9.3 |
| Social relations | | |
| Frequency/type (score)¹ | 9.1 | 10.6 |
| Relationship (D-T scale)¹ | 13.6 | 13.2 |
| Finances | | |
| Enough money (score)¹ | 3.9 | 3.6 |
| Money available (D-T scale)¹ | 12.7 | 12.1 |
| Health | | |
| General well-being¹ | 13.1 | 12.5 |
| Feelings about health (D-T scale)² | 8.9 | 7.9 |
| EQ-5D² | | |
| Health state score | 57.7 | 59.5 |

CGI, Clinical Global Impression;
D-T, ‘delighted-terrible’;
EQ-5D, EuroQoL EQ-5D;
GAF, Global Assessment of Functioning;
PANSS, Positive and Negative Syndrome Scale;
QoL, quality of life.
¹Independent t-test not significant at P = 0.05.
²Significant at P < 0.05 (similar melts achieved using non-parametric tests).

Using the Lehman ‘delighted-terrible’ (D-T) scale and scores, relapse patients appeared to experience lower QoL than non-relapse patients on most dimensions, but the differences were small and not statistically significant, except for the items ‘living arrangements’ and ‘feelings about current health’ (P<0.05). There was perhaps some inconsistency in the QoL findings since relapse patients scored slightly better on the EQ-5D visual analogue scale compared with non-relapse patients (P<0.05). However, the EQ-5D measures own health state today, whereas the Lehman score covers broader dimensions of quality of life.

Resources and Costs

Six-month service use rates and costs per patient are summarized in TABLE 5. Costs for relapse cases were four times higher than those for non-relapse cases—£8212 compared with £1899 (P<0.05)—with much of the cost difference accounted for by in-patient days. During the 6 months prior to the study, patients in the relapse group spent a mean of 58 days in hospital—although this figure was inflated by six patients who were continuously in hospital for the entire period. By design and selection, nobody in the non-relapse group experienced any hospitalization in this period.

TABLE 5
Mean 6-month service use and costs
(£, 1998) per patient by relapse status

| | Non-relapse | | Relapse | |
|---|---|---|---|---|
| | (n = 68) | | (n = 77) | |
| | Mean | Costs | Mean | Costs |
| Service | usage | (£) | usage | (£) |
| In-patient care (days)¹ | 0.0 | 0 | 57.8 | 6451 |
| Out-patient | | | | |
| Psychiatric visits¹ | 1.4 | 135 | 2.1 | 209 |
| Other² | 0.1 | 8 | 0.3 | 19 |
| Day hospital (visits)² | 2.3 | 133 | 2.1 | 126 |
| Community mental health centre (visits)^{2,3} | 2.4 | 44 | 1.4 | 25 |
| Day care Centre (visits)¹ | 5.9 | 106 | 0.9 | 15 |
| Group therapy^{2,3} | 0.4 | 6 | 0.1 | 2 |
| Sheltered workshop³ | 1.1 | 45 | 0.0 | 0 |
| Specialist education^{2,3} | 2.9 | 52 | 0.0 | 0 |
| Other (not specified)³ | 0.6 | 12 | 0.0 | 0 |
| Visits by | | | | |
| Psychiatrist¹ | 2.5 | 103 | 2.3 | 269 |
| Psychologist | 0.0 | 0 | 0.0 | 2 |
| General practitioner³ | 1.8 | 217 | 1.6 | 152 |
| District nurse³ | 0.1 | 1 | 0.0 | 0 |
| Community psychiatric nurse³ | 12.6 | 1014 | 5.2 | 791 |
| Social worker³ | 0.1 | 24 | 0.4 | 106 |
| Occupational therapist³ | 0.0 | 1 | 0.8 | 44 |
| Home help/care worker³ | 0.4 | 0 | 0.6 | 0 |
| Total costs¹ | | 1899 | | 8212 |

¹Independent t-test significant at P < 0.05 (similar results achieved using non-parametric tests).
²Costs not available - set equal to cost for day care centre.
³Independent t-test not significant at P = 0.05.

Psychiatric out-patient visits were also significantly more common in relapse than in non-relapse cases (mean cost £209 v. £135, P<0.05). On the other hand, there was slightly higher use by patients in the non-relapse group of day care centers, group therapy, sheltered workshops, specialist education, general practitioners and Community Psychiatric Nurse (“CPN”) visits, but apart from day care centers none of the differences was statistically significant at the 5% level. Services are complements, in the sense that patients with greater morbidity are likely to use more of a number of services, but are also substitutes, in that (for example) hospital in-patients will have less need and less opportunity to use day care, primary care and CPN support. These two tendencies may have cancelled out for this sample.

Relapse Correlates

Given the (expected) high costs associated with illness relapse, correlates of relapse and non-relapse status were examined. The odds ratios in TABLE 6 indicate that, controlling for all other explanatory factors, there was an increased risk of relapse associated with:

(a) each year of age (OR=1.07);
(b) fewer years since recent hospital admission (converting the tabulated OR: 1/0.79=1.27);
(c) previous suicide or self-harm attempts (OR=3.93);
(d) increased social functioning (OR=1.29); and
(e) lower scores on the GAF (converting the tabulated OR: 1/0.93=1.08) (all P<0.05).

TABLE 6
Factors assciciated with relapse status: multivariate analysis (n = 131)¹

| Variable | Odds ratio² | 95% CI |
|---|---|---|
| Age (years) | 1.07 | 1.01-1.13 |
| Number of years since most recent | 0.79 | 0.69-0.90 |
| hospital admission | | |
| Previous suicide or self-harm attempts | 3.93 | 1.39-11.07 |
| Social relationships score (Lehman) | 1.29 | 1.13-1.48 |
| GAF score | 0.93 | 0.87-0.98 |

GAF, Global Assessment of Functioning.
¹Dispersion parameter 0.99 (a value of 1 indicates constant variance or the error term).
²Significant at P < 0.05 controlling for gender, ethnicity, marital status, education and living arrangemets (all P > 0.05).

Cost Correlates

The log link method of GLM estimation was used to examine the factors associated with cost differences, as shown in TABLE 7. Coefficient values represent the percentage change in total costs (from the average) following a one-unit change in the explanatory variable (compared with a reference category if the variable is categorical). Holding constant all other explanatory factors in the model, average costs were increased by patients who relapsed (147%), and were reduced by patients who were older (3.6% per year of age), and living with family/others compared with those in collective accommodation (58%).

TABLE 7
Factors associated with differences in
costs multivariate analyses (n = 145)

| Variable | Coefficient (β)¹ | 95% CI |
|---|---|---|
| Age (years) | −0.04 | −0.06 to −0.16 |
| Gender (male) | 0.08 | −0.32 to 0.48 |
| Ethnicity (White) | −0.11 | −0.64 to 0.43 |
| Ethnicity (Black Caribbean) | 0.99 | −0.15 to 2.12 |
| Marital status (single) | −0.16 | −0.70 to 0.38 |
| Marital status (married/cohabiting) | 0.35 | −0.33 to 1.03 |
| Further education (higher) | 0.26 | −0.44 to 0.94 |
| Living alone at home | −0.05 | −0.58 to 0.48 |
| Living with family/relatives | −0.58 | −1.07 to −0.08 |
| Relapse status | 1.47 | 1.88 to 1.06 |
| Constant | 9.15 | 8.07 to 10.14 |

¹Percentage change in total costs following a one-unit change in the explanatory variable; all variables significant at P < 0.05.

Discussion

Costs of Relapse of Schizophrenia

Studies of the overall costs of schizophrenia in the United Kingdom and in other countries confirm the high proportion of the total that is attributable to in-patient care. This study shows that illness relapse is a major factor in generating these high hospitalization rates and costs. Patients who experienced a relapse during the 6 months prior to data collection had mean service costs of £8212 compared with £1899 for those who had no relapse during this period. The only previous United Kingdom estimate of the costs of relapse of which awareness existed was based on expert opinion and assumed (rather than observed) service utilization in a simulation model that compared three antipsychotic drugs. Average relapse costs at 1997 prices were estimated to be just over £10 000 per patient during three monthly cycles and included both service use costs and accommodation costs (the latter not included here).

Clinical and QoL Correlates

Surprisingly, perhaps, there were few differences in clinical and QoL outcomes between patients who had relapsed and those who had not. However, some of the patients in the former group would have recovered well from their relapse by the time these clinical and QoL instruments were administered. This time lapse is probably the reason for the lack of difference.

Associations

Multivariate analyses confirmed some significant correlates of relapse, and a reduced-form cost equation found, as expected, that relapse status significantly increased total costs. The cost equation was estimated in reduced form for two main reasons. First, relapse status as a regressor captured some of the important partial effects already identified in the relapse function—for example, suicide attempts, previous hospital admissions and social functioning—and reduced the need to include these variables further as independent effects in the cost analyses. Second, clinical and QoL variables were excluded from the cost equation because it was difficult to relate current measures with costs in the previous 6 months. This is a problem of endogeneity. It is difficult to ascertain the direction of causation between variables. Although higher levels of service use (and costs) might have improved health and reduced the likelihood of relapse, relapse status might have increased service use and costs. However, given that relapse often resulted in hospitalization (for about two-thirds of the people in the relapse group) and in-patient costs accounted for around three-quarters of total costs, the problem of endogeneity with relapse status was less of an issue.

Finally, a cautionary note is required on measuring differences in costs and health outcomes between the relapse and non-relapse groups. Although this method is valid, a superior comparison would come from panel or longitudinal data that measure changes in outcomes prospectively for a given population. The costs of relapse would then be estimated by examining the differences in costs, before, during and after relapse. Cost-effectiveness comparisons are also required based on experimental evaluations of relapse minimization strategies.

Policy Implications

The significant costs found to be associated with relapse confirm the scale of the impact—in this case measured by service uptake—of a worsening of symptoms for people with schizophrenia. These costs will be of interest to clinicians and other decision-makers who face difficult choices about new but more expensive treatments for patients with schizophrenia. Subject to the above cautionary comment, delaying the time to relapse should mean delaying the escalation of costs. More importantly, a slower or reduced rate of relapse means slower or reduced damage to the health and quality of life of patients, and in some cases also less adverse impact on their families.

Psychoeducation and related programs have been shown to reduce medication non-adherence, detect prodromal symptoms of relapse and reduce the rate of hospitalization. A relatively inexpensive evidence-based intervention for reducing relapse is family work for patients with schizophrenia living with a relative with high levels of expressed emotion. There is no evidence that these effective interventions have yet come into widespread use.

If new antipsychotic treatments in schizophrenia can improve efficacy and compliance rates compared with conventional neuroleptic therapy, and thereby reduce relapse rates, this might bring about reductions in the service costs of schizophrenia. In turn, as demonstrated in some international studies, and as concluded by the National Institute for Clinical Excellence (2002), the overall costs of the treatment could be reduced.

Illustrative Computing System(s)

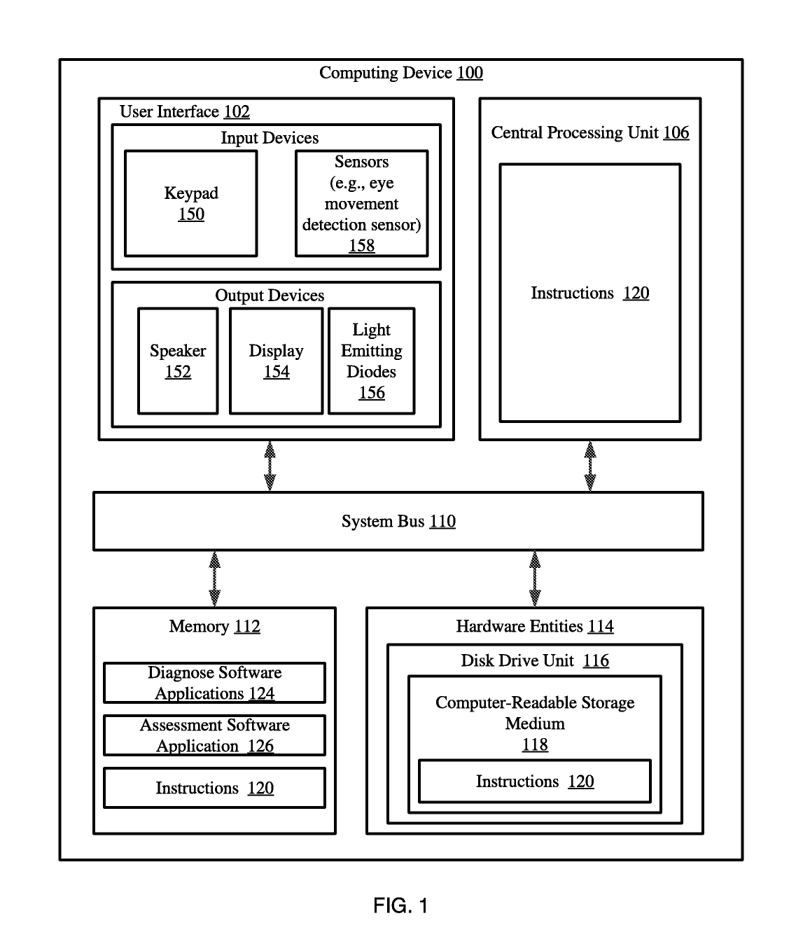

Referring now to FIG. 1, there is provided a detailed block diagram of an illustrative architecture of a computing system 100. Notably, the computing system 100 may include more or less components than those shown in FIG. 1. However, the components shown are sufficient to disclose an illustrative embodiment implementing the present solution. The hardware architecture of FIG. 1 shows an example of a computing system configured to facilitate the provision of diagnosing and assessing therapeutic efficacy of schizophrenia. As such, the computing system 100 of FIG. 1 implements at least a portion of the methods described herein.

The computing system 100 includes any type of computing device. For example, the computing system 100 includes, but is not limited to, a desktop computer, a laptop computer, a personal digital assistant, a mobile phone, a smart phone, and/or a tablet computer.

Some or all the components of the computing system 100 may be implemented as hardware, software and/or a combination of hardware and software. The hardware includes, but is not limited to, one or more electronic circuits. The electronic circuits may include, but are not limited to, passive components (e.g., resistors and capacitors) and/or active components (e.g., amplifiers and/or microprocessors). The passive and/or active components may be adapted to, arranged to and/or programmed to perform one or more of the methodologies, procedures, or functions described herein.

As shown in FIG. 1, the computing system 100 includes a user interface 102, a processor, e.g., a central processing unit (“CPU”) 106, a system bus 110, a memory 112 connected to and accessible by other portions of computing device 100 through system bus 110, and hardware entities 114 connected to system bus 110. The user interface 102 may include input devices (e.g., a keypad 150 and/or sensors 158) and output devices (e.g., speaker 152, a display 154, and/or light emitting diodes 156), which facilitate user-software interactions for controlling operations of the computing system 100.

At least some of the hardware entities 114 perform actions involving access to and use of memory 112, which may be a Random Access Memory (“RAM”), a disk drive and/or a Compact Disc Read Only Memory (“CD-ROM”). Hardware entities 114 may include a disk drive unit 116 comprising a computer-readable storage medium 118 on which is stored one or more sets of instructions 120 (e.g., software code) configured to implement one or more of the methodologies, procedures, or functions described herein. The instructions 120 may also reside, completely or at least partially, within the memory 112 and/or within the CPU 106 during execution thereof by the computing system 100. In some scenarios, different portions of instructions 120 may be stored in components 106, 112, 114. The memory 112 and the CPU 106 also may constitute machine-readable media. The term “machine-readable media”, as used here, refers to a single medium or multiple media (e.g., a centralized or distributed database, and/or associated caches and computer devices) that store the one or more sets of instructions 120. The term “machine-readable media”, as used here, also refers to any medium that is capable of storing, encoding or carrying a set of instructions 120 for execution by the computing system 100 and that cause the computing system 100 to perform any one or more of the methodologies of the present disclosure.

In some scenarios, the hardware entities 114 include an electronic circuit (e.g., a processor and/or a graphics card) programmed for facilitating the diagnosis and assessment of therapeutic efficacy of schizophrenia. In this regard, it should be understood that the electronic circuit may access and run diagnose software applications 124 and assessment software applications 126 installed on the computing system 100. The software applications 124-126 are generally operative to facilitate the diagnosing and assessment of therapeutic efficacy of schizophrenia. Other functions of the software applications 124-126 will become apparent from other portions of the discussion presented herein.

The present solution is not limited to scenarios in which a single computing device is employed for implementing the methods described herein. In some scenarios, a network-based system may be employed comprising at least two computing devices communicatively coupled to each other via a wired and/or wireless connection. One of these computing devices may include, but is not limited to, a server for accessing, storing and retrieving data stored in a data store (e.g., a database). If medical related information is communicated over a network link, then the encryption may be employed to encrypt the medical related information prior to such communication. Any known or to be known encryption technique may be used herein without limitation.

Illustrative Method(s)

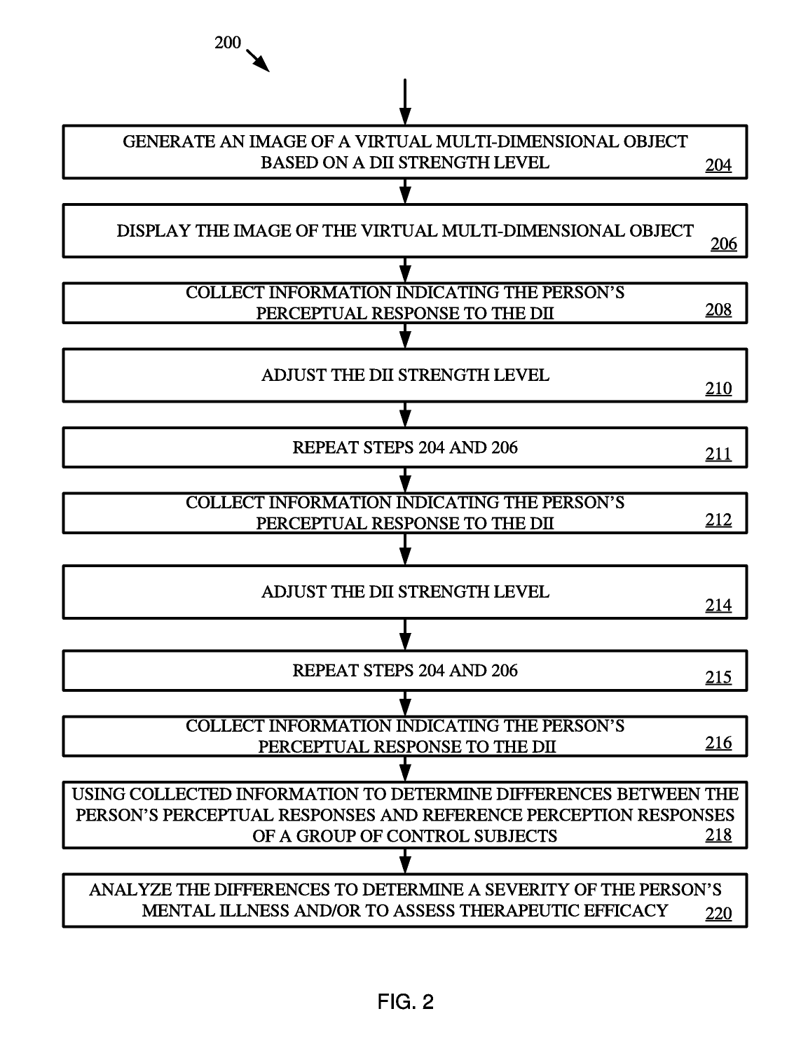

Referring now to FIG. 2, there is provided an example of a flow diagram of a process 200 for determining a severity of a person's mental illness and/or assessing therapeutic efficacy. The process 200 may include generating an image of a virtual multi-dimensional object based on a DII strength level 204. A DII strength level is used in generating the image of the virtual multi-dimensional object testing a person's susceptibility to a DII. DII occurs when concave objects appear convex. The object may include, but is not limited to, a 3D hollow mask object that is perceptible as a convex mask and/or a concave mask. In the 3D hollow mask scenarios, a visual illusion in which the perception of a concave mask of a face appears as a normal convex face. This visual illusion is referred to herein as Hollow Mask Illusion or HMI. This step will be described in detail later.

The process 200 also includes rendering the image of the virtual multi-dimensional object on a display screen 206. The display screen may be the display of the computing system (e.g., display 154 of FIG. 1) a display screen communicatively coupled to the computing system. Alternatively, and/or additionally, the process may repeatedly generate a sequence of images of the virtual multi-dimensional object using the step of 204, each image corresponding to a view angle of the virtual multi-dimensional object, and render the sequence of images in an animation on the display screen.

The animation may be designed to show the virtual multi-dimensional object in a plurality of successive positions to create an illusion of movement. For example, in the face mask scenarios, the animation shows the face mask rotating a certain distance about a central vertical axis in a first direction, followed by the rotation thereof in a second opposing direction. The present solution is not limited to the particulars of this example. Any type of movement may be shown via the animation. However, rotational movement has been shown to have certain advantages in the present application since this type of movement allows a person to recover the 3D structure of the object.

The process 200 also includes collecting information indicating the person's perceptual response to the DII 208. This information may include, but is not limited to, sensor data specifying tracked eye movements and/or user-input information specifying the person's answers to questions. The sensor data may be generated by one or more sensors (e.g., sensors 152 of FIG. 1) coupled to or part of the computing device. The person may be prompted to perform a user-software interaction to answer a question. For example, the person is prompted to answer the following question: “Is the mask concave, convex or flat?”. The user-software interaction may be achieved via a depression of a key on a keyboard (e.g., keypad 150 of FIG. 1), a virtual button presented on a touch screen (e.g., display 154 of FIG. 1), and/or voice recognition. In some scenarios, a stereoscopic display is employed.

The process 200 may further include adjusting the DII strength level to a second strength level 210 and repeating the steps of 204, 206 (i.e. 211). For example, the process may increase the DII strength level. Subsequently, the process may again collect information that indicates the person's perceptual response to the DII, as shown by 212. This information may be collected in the same or substantially similar manner as that collected in previous 208. Optionally, the above process may further adjust the DII strength level 214, e.g., decrease the strength level and repeat the steps of 204, 206 (i.e., 215). Subsequently, the process may again collect information that indicates the person's perceptual response to the DII, as shown by 216. This information may be collected in the same or substantially similar manner as that collected in previous 208 and/or 212. The increase or decrease of the DTI's strength level causes a textured state of the virtual multi-dimensional object to be transformed from a first textured state to a second different textured state to provide a DII with a higher or lower illusion strength level. This step of adjusting the DII strength level will be described later in detail.

The process 200 further uses the collected information above to determine differences between the person's perceptual responses and reference perception responses of a group of control subjects to the DII 218. The mapping operations involve respectively mapping the differences between the person's perceptual responses to the DII and reference perception responses (e.g., average perceptual responses of a group of controls, namely individuals absent of any mental illness) to the same DII.

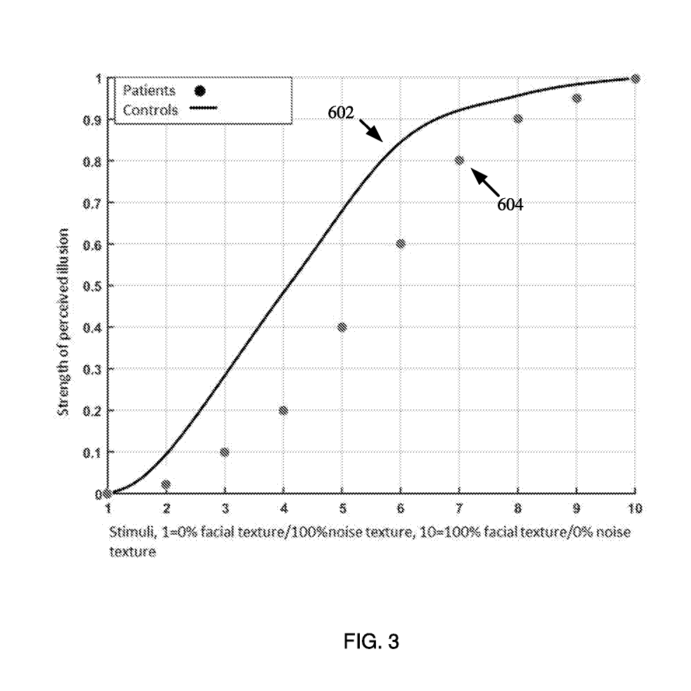

An illustrative map is shown in FIG. 3. The map comprises a graph having a two-dimensional coordinate system (e.g., an x-y coordinate system) onto which a plurality of data points have been plotted. The system may plot first data points on a graph having a two-dimensional coordinate system, the first data points representing the person's perceptual responses to the DII at a range of DII strength levels. The system may also plot second data points representing perception responses of a group of control subjects to the DII at the range of DII strength levels. The system may respectively compare the first data points to second data points representing perception responses of a group of control subjects to the DII at the range of DII strength levels to determine the differences on a cumulative basis, which will be explained in detail later. Larger values of cumulative differences indicate more acute stages of the disease.

As shown in FIG. 3, the x-axis lists the stimuli (e.g. an image of a hollow mask with a DII strength level) that were used during the method. The y-axis comprises values specifying the perceived strength of the DII for the corresponding stimuli. The solid line 602 represents a curve that has been obtained from the data of perception responses of a group of control subjects. The collected information is then used to plot the solid dots 604 representing the person's perceptual responses to the DII at different strength levels, which processed is explained in detail as below.

In collecting the responses, the system may display a small number of masks (e.g., 10) that are pre-selected, based on pilot data with control subjects, to cover the range from very weak to very strong DII illusions. In collecting the responses, the system shows each stimulus for a number of times and records the responses in a digital representation. In a non-limiting example, the system may repeat the presentation of each stimulus for a number of times (e.g., 6 times), and record the responses in a digital representation. For example, the system may code the digital representation with a binary sequence, such as A=[0 1 1 1 0 1], where A stands for stimulus, 1 stands for being perceived as concave and 0 for being perceived as convex. Based on the digital representation of the response, the subject perceived the stimulus A two times as convex and four times as concave. Focusing on the concave responses: this means that the subject perceived stimulus A 66.7% of the time as concave. The data point on the y-axes should indicate 0.667 or 66.7% as the strength of the perceived illusion. Convex stimuli are used as “catch trials” because it is known that convex stimuli should be perceived as convex more than 95% of the time. The critical responses are those to the concave stimuli.

In some scenarios, the system determines the y-axis values as follows. For each mask, the corresponding y-axis value is the percentage of times that the mask was perceived as convex divided by the total number that this particular mask was presented. The system plots these data as individual dots 604 as shown in FIG. 3, and display the solid line 602 as the average performance of healthy controls.

In some scenarios, in determining the differences between a patient's responses and the control group, the system may determine first data points representing the person's perceptual responses to the DII at a range of DII strength levels, determine second data points representing perception responses of a group of control subjects to the DII at the range of DII strength levels, respectively compare each of the first data points to a corresponding data point in the second data points to determine a difference, and determine the differences by accumulatively adding the difference for each of the first data points over the range of the DII strength levels. For example, the magnitude of the differences for a patient may be represented as:

M=Σ(Ck−Sk)

where the sum is from k=1 to k=N, N is the number of masks, Sk is the y-axis value for mask k for the patient and Ck is the corresponding y-axis value for mask k for healthy controls.

Returning to FIG. 2, optionally, the process 200 may analyze the differences in 220 to (a) determine a severity of the person's mental illness (e.g., schizophrenia) and/or (b) to assess therapeutic efficacy. For example, the differences between the y-axis values of the first data points and the second data points at each stimulus 1-10 on the y-axis of FIG. 3 are determined and used to (a) determine a severity of the person's mental illness (e.g., schizophrenia) and/or (b) to assess therapeutic efficacy.

Details on how reduced depth inversion illusion varies with hospitalization and with disease severity is documented in Keane, B. P., Silverstein, S. M., Wang, Y. S., Papathomas, T. V.: Reduced depth inversion illusions in schizophrenia are state specific and occur for multiple object types and viewing conditions. J. Abnorm. Psychol. 122(2), 506-512 (2013). doi: 10.1037/a0032110, which is incorporated by reference.

In some or other scenarios, the system may vary the strength level of the DII and examine changes in a patient's responses to the different strength of the DII. In other words, the system may use different levels of DII strength to assess changes in perceptual responses (comparing results to control population, comparing results before/after treatment) in relation to disease severity or elapsed time since last admission to partial acute hospitalization. In a non-limiting example, the system compared 30 Schizophrenia patients and 25 well-matched healthy controls on the perceived strength of the hollow mask illusion. The experiment used physical objects (masks and scenes). Results showed that patients experienced fewer illusions than controls. In addition, patients' veridical perception rates (seeing the concave mask as concave) increased with positive symptoms. Results also showed that patients' veridical perception rates decreased with time elapsed since last acute partial hospital admission. Based on the above empirical results, the system may be configured to assess the therapeutic efficacy or disease severity by detecting an indication of either an increase or a decrease in veridical responses to a 3-D hollow mask. For example, if the system determines that the veridical responses have increased, the system may determine that the severity of the disease has also increased.

Notably, the present solution is not limited to the particular method, such as 200 shown in FIG. 2. For example, a subject's response may be recorded in relation to any number of DII strength levels, not just three. In some scenarios, the process is performed for each DII strength level with a plurality of stimuli (e.g., 10 stimuli) that give rise to illusory percepts of various degrees of illusion strength.

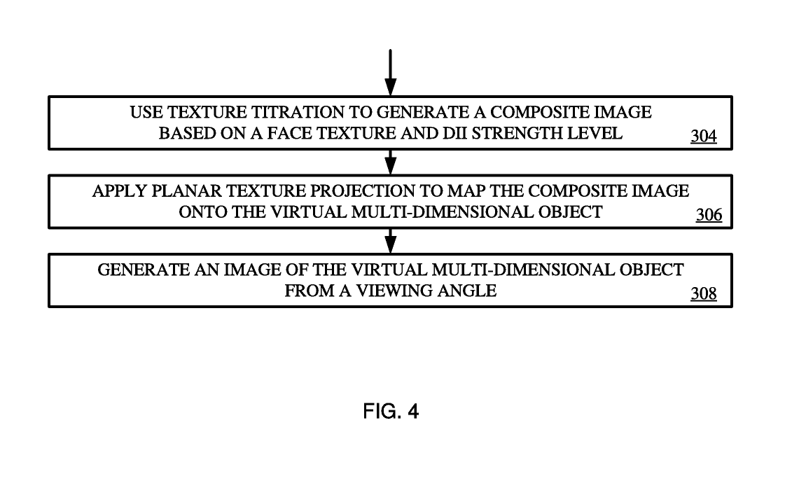

Now, with reference to FIG. 4, the step of generating an image of a virtual multi-dimensional object (204 in FIG. 2) is further explained. The step includes: using texture titration to generate a composite image based on a face texture image and the DII strength level 304; applying planar texture projection to map the composite image onto the virtual multi-dimensional object to generate a mapped 3-D model 306; and generating the image of the virtual multi-dimensional object based on a projected view of the mapped 3-D model from a viewing angle 308.

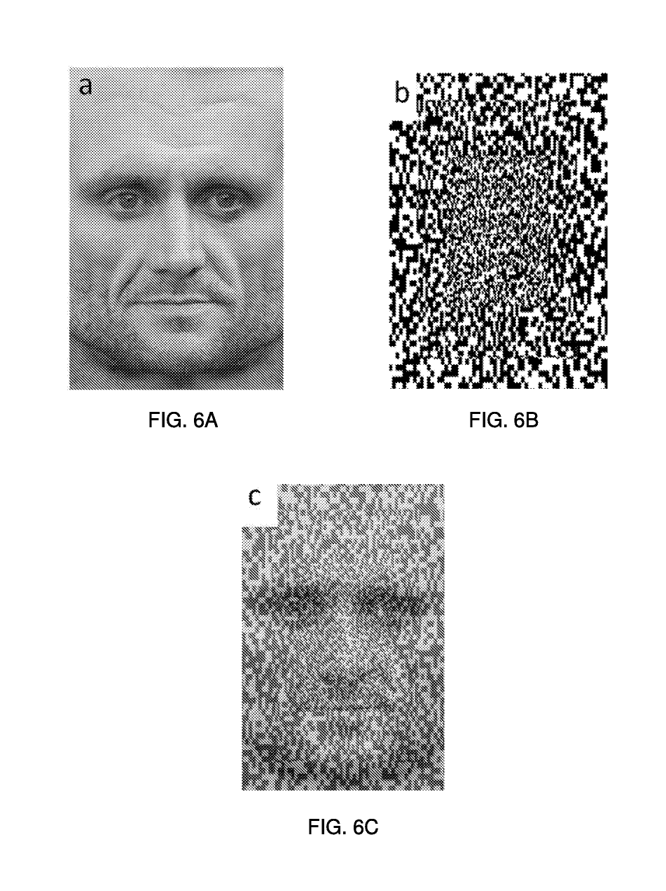

In some scenarios, a face texture in step 304 may be generated by: creating a computer model of a human head; removing certain features of the human head (e.g., teeth, tongue, hair, eyelashes, etc.) from the computer model; dividing the head of the computer model in two portions (e.g., a front portion and a back portion); and adjusting the size of remaining facial features (e.g., nose) of the front portion of the computer modeled head. An example of a face texture is shown in FIG. 6A.

Returning to FIG. 4, in step 304, using texture titration may include mapping texture onto the front portion of the computer modeled head in a pixels-by-polygon manner. Methods for creating computer models of multi-dimensional object and adding texture to the computer modeled multi-dimensional objects are well known in the art. Any known or to be known method for creating computer models of multi-dimensional object and adding texture to the computer modeled multi-dimensional objects may be used herein without limitation. Such methods are implemented in Illustrator tools, such as Adobe Illustrator. Any known or to be known Illustrator tool may be used here to generate the virtual multi-dimensional object.

The texture may include, but is not limited to, a skin texture, a wood grain texture, a mosaic texture, a patchwork texture, a stained glass texture, a craquelure texture, a fabric texture, a metal texture, a random-dot texture, and/or a plastic texture. The texture may be selected in accordance with any given application. The texture is at least partially defined by a surface roughness, a surface color, surface density and irregularities of the virtual object. The texture may be modified by adjusting the value of a noise/grain parameter, a color parameter, a density parameter, a roughness parameter, and/or other parameters.

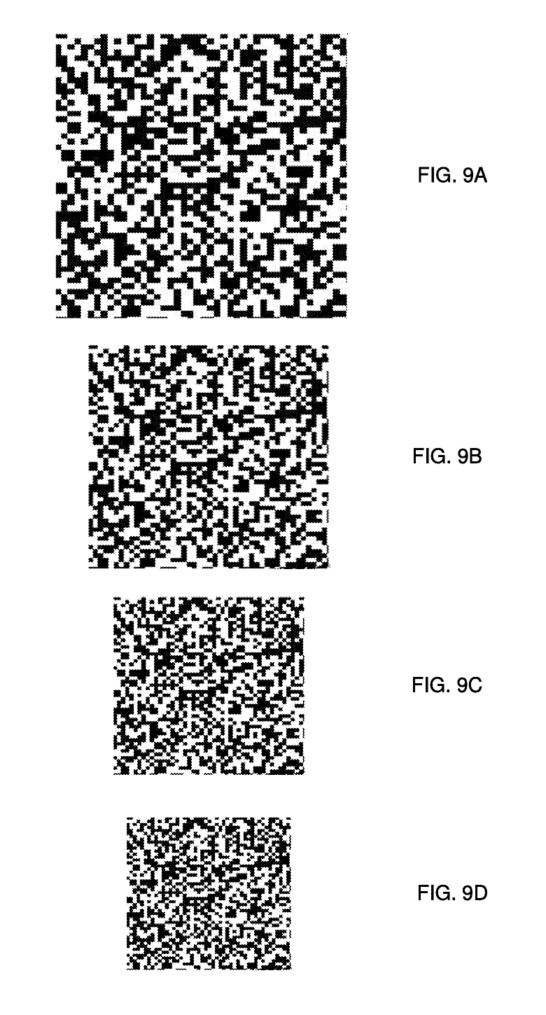

With reference to FIG. 9A, an example of a random-dot texture is shown. In generating the random-dot texture, the computing system may generate an image array that includes multiple units, assign values of [0,1] (white or black) to each unit with equal probability (such as randomly selecting 0 or 1 for each unit), and resize the image with a scaling factor to a desired image size. For example, the system may generate an 80×80 image array, each unit in the array is randomly assigned a value of 0 or 1 with equal probability. The system may further scale up the image array by a scaling factor of 6.4 to generate a random-dot texture that has a size of 512×512 (as shown in FIG. 9A).

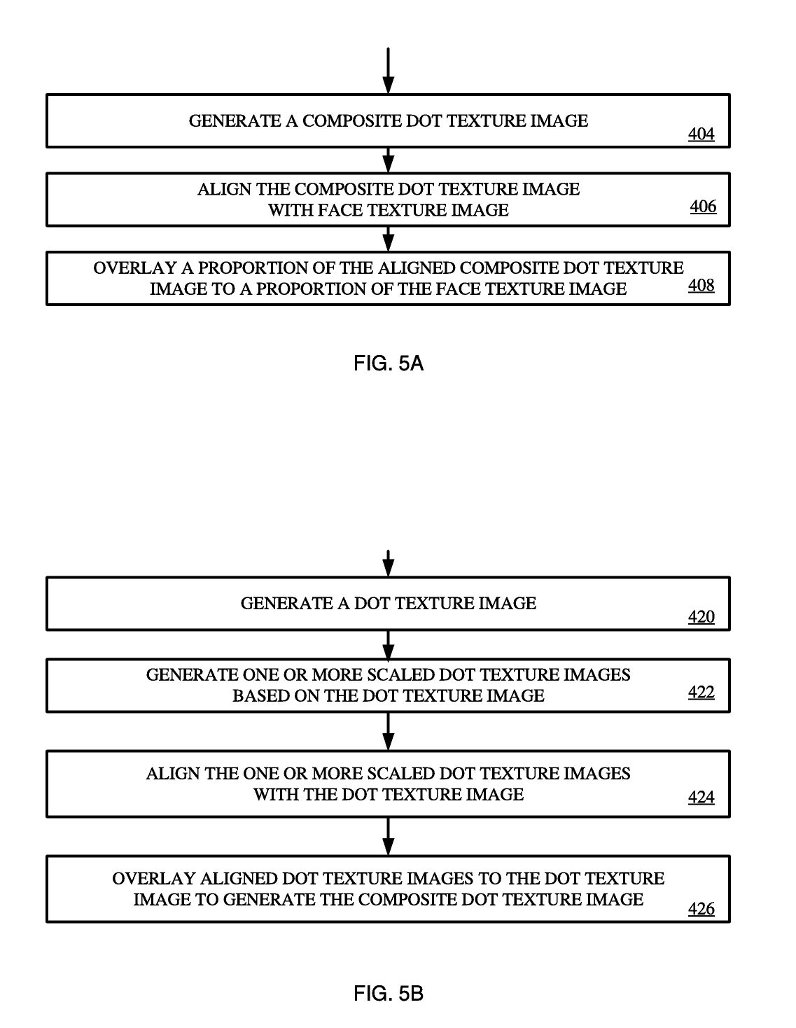

With reference to FIG. 5A, the step for generating the composite image (304 in FIG. 4) is now further explained. The process for generating the composite image may include: generating a composite dot texture image 404; aligning the composite dot texture image with the face texture image 406; and overlaying a first proportion of the aligned composite dot texture image to a second proportion of the face texture image 408. In some scenarios, the first and second proportions are summed at a value of one. The process described in FIG. 5A can be further represented by an equation:

imgC(i,j)=f1*imgF(i,j)+f2*imgR(i,j), Equation (1)

where imgC(i,j) is the composite image, imgF(i,j) is the face texture (an example is shown in FIG. 6A), imgR(i,j) is the texture image (such as the composite dot texture image, which is to be further explained), f1 and f2 respectively represents the first and second proportion. In some scenarios, 0<=f1, f2<=1 and f1+f2=1.

Now, generating a composite dot texture image is further described, with reference to FIG. 5B. In FIG. 5B, a process for generating the dot texture image may include: generating a dot texture image 420 (an example of a random-dot texture image is shown in FIG. 9A and explained above); generating one or more scaled dot texture images based on the dot texture image 422, wherein each scale dot texture image is scaled down a percentage from the dot texture image; aligning the one or more scaled dot texture images with the dot texture image 424; and overlaying the one or more aligned dot texture images to the dot texture image to generate the composite dot texture image 426. In step 426, each pixel in the composite dot texture image has a value of black if at least one corresponding pixel in the dot texture or the one or more aligned dot texture images has a value of black; otherwise the pixel in the composite dot texture image has a value of white.

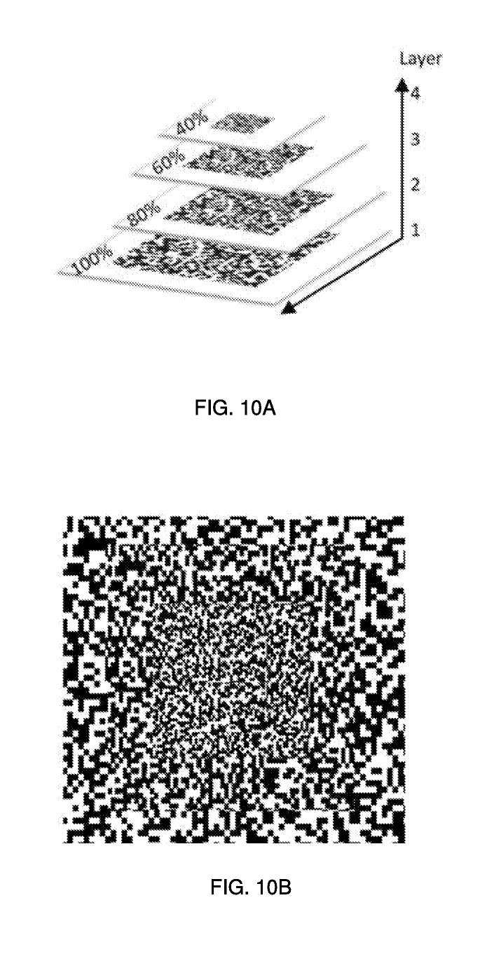

In a non-limiting example, FIGS. 9A-9D show a series of consecutively scaled dot texture images. FIG. 9A is a full-scale dot texture image as explained above. FIGS. 9B, 9C and 9D are respectively scaled down from the image in FIG. 9A by 20%, 40% and 60%. FIG. 10A shows that the images from FIGS. 9A-9D are further aligned, for example, at their centers, and overlaid by layering to produce the composite dot texture image. FIG. 10B shows the composite dot texture image. As shown in FIG. 10B, the gradual scaling of textural elements as a function of their distance from the center (via overlay) suggests the depth structure

Returning to FIG. 5A and Equation (1) above, the composite dot texture image (such as shown in FIG. 10B, which is also scaled as shown in FIG. 6B) and the face texture image (such as shown in FIG. 6A) are aligned and overlaid to produce the composite image in FIG. 6C. In aligning the composite dot image and the face texture image, the system may scale the size of the composite dot image to that of the face texture image. For example, the size of the face texture image (in FIG. 6A) is 300 (width) by 420 (height) in pixels. The system may scale the composite dot image (as in FIG. 10B) from its size 512×512 to 300×400, as shown in FIG. 6B, overlay the face texture image and the scaled composite dot texture image to produce the composite image, as shown in FIG. 6C.

In overlaying the composite dot texture image and the face texture image, the proportions of each image in the step of overlay 408 are represented by Equation (1), where f1 and f2 (f1+f2=1), and can be adjusted. In some scenarios, f1 or f2 can be used to represent the strength level of the DII. For example, FIG. 6C is generated using f1=45% (for face texture image) and f2=55% (for composite dot texture image). This is an example of texture titration that is expected to elicit a DII with a medium illusion strength. Changing the values of proportions f1 and f2 may change the DII strength level, as will be further explained later.

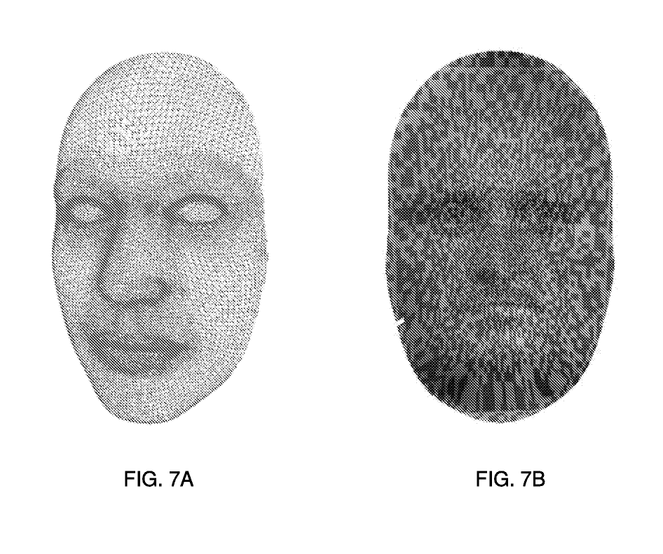

Now, returning to FIG. 4, the steps 306, 308 are further explained with examples. In step 306, the composite image (as shown in FIG. 6C and explained above) is mapped to a virtual multi-dimensional object (shown in FIG. 7A) to generate a mapped 3-D model of the multi-dimensional object. As shown in FIG. 7A, the virtual multi-dimensional object is a gender-neutral 3-D mask (shown as a mesh generated by a computer graphics software). The system may use any suitable planar texture projection algorithm, now or later developed. In some scenarios, the system may map the composite image onto both concave and convex sides of the virtual multi-dimensional object to generate the mapped 3-D model. Alternatively, the system may map the composite image on either concave or convex side of the object. In the case of 3-D face mask, convex side refers to the side of the mask with the nose sticking towards the viewer. Concave side refers to the opposite of the convex side with the nose sinking in and is further away from the viewer. An example of the resultant image from step 308 is shown in FIG. 7B, which corresponds to a view of the mapped 3-D model from the front, i.e. convex side.

The concave side of the mask, when mapped with the composite image (e.g., via steps 304-308) and viewed by a patient with schizophrenia, it may be perceived as convex or opposite (i.e. concave) depending on the strength level of the DII and the severity of the mental illness. As described above, the DII strength level can be adjusted by changing the proportion of the composite dot texture image and the proportion of the face texture image in overlaying the two images (step 408 in FIG. 5A and Equation (1)).

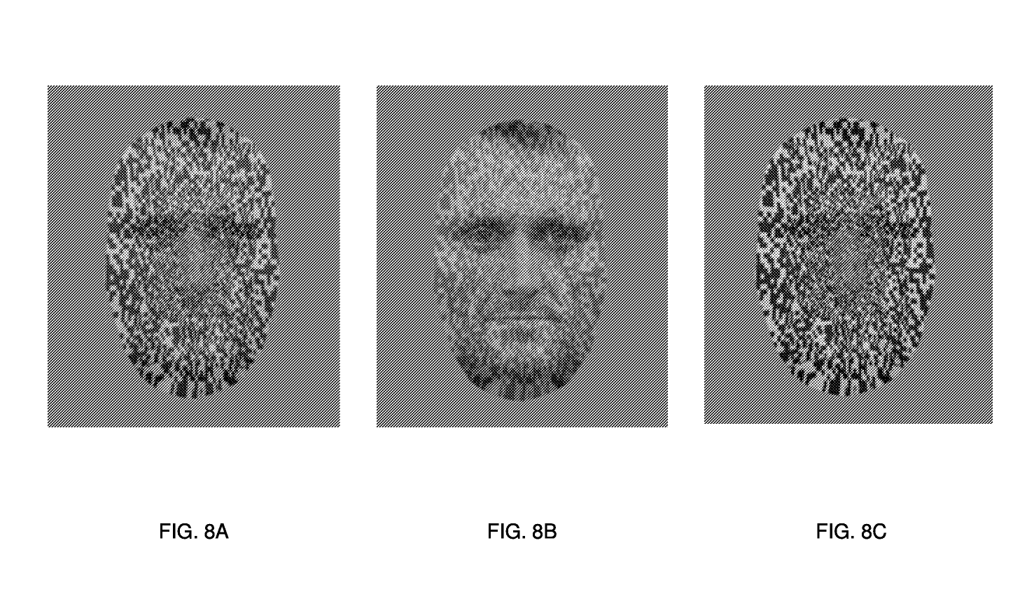

In a non-limiting example, FIG. 8A is illustrative of an image of virtual multi-dimensional object that results from a concave mask with a 45% facial texture and a 55% random dot texture. This example of texture titration elicits a DII with medium illusion strength. The steps of adjusting the DII strength level (210, 214 in FIG. 2) are also further explained with examples shown in FIGS. 8B and 8C. In FIG. 8B, the virtual multi-dimensional object comprises a concave mask with a 75% facial texture and a 25% random dot texture. This is done by increasing the DII strength level by decreasing f2 (or increasing f1) in Equation (1), which causes the textured state of the virtual multi-dimensional object is transformed from a first textured state to a second different textured state. In this case, random noise texture is removed from the multi-dimensional object such that its textured state is changed from a first noisy textured state to a second less noisy textured state. This example of texture texturing is expected to increase the DII strength level.

In FIG. 8C, the virtual multi-dimensional object comprises a concave mask with a 30% facial texture and a 70% random dot texture, which can be done by decreasing the DLL strength level by increasing 12 (or decreasing f1) in Equation (1), causing the multi-dimensional object to be transformed to a noisier texture state. This example of texture titration is expected to decrease the DII strength level.

The above illustrated systems and methods provide advantages over the prior art in that they use computer graphics techniques to generate one or more images of a virtual multi-dimensional object at various DII strength levels that are suitable for diagnosing and assessing therapeutic efficacy of schizophrenia. These advantageous are achieved via various steps described above. For example, the system generates a random-dot texture (such as shown in FIG. 9A) in a particular density of elements (e.g., 80×80 image array) that aid the perceptual system in breaking the illusion (i.e. the concave mask will be perceived as concave). Other density for the random-dot texture may be used. The system also generates a composite dot texture image by aligning and overlaying a series of scaled dot texture images (such as shown in FIG. 5B). The number of scaled dot texture images in the overlay is four in the example shown. However, more or fewer scaled dot texture images may be used. The system also generates a composite image by overlaying the composite dot texture image with a face texture image (such as shown in FIG. 5A) at various proportions to achieve various DIIs that correspond to different DII strength levels. The system further applies planar texture projection to map the composite image onto the virtual multi-dimensional object to generate one or more images of the virtual multi-dimensional object and form stimuli in the test. All these methods and other illustrated methods help achieve the advantages over prior art systems.

All of the apparatus, methods, and algorithms disclosed and claimed herein may be made and executed without undue experimentation in light of the present disclosure. While the present solution has been described in terms of preferred embodiments, it will be apparent to those having ordinary skill in the art that variations may be applied to the apparatus, methods and sequence of steps of the method without departing from the concept, spirit and scope of the present solution. More specifically, it will be apparent that certain components may be added to, combined with, or substituted for the components described herein while the same or similar results would be achieved. All such similar substitutes and modifications apparent to those having ordinary skill in the art are deemed to be within the spirit, scope and concept of the present solution as defined.

The features and functions disclosed above, as well as alternatives, may be combined into many other different systems or applications. Various presently unforeseen or unanticipated alternatives, modifications, variations or improvements may be made by those skilled in the art, each of which is also intended to be encompassed by the disclosed solutions.

We claim:

1. A method for diagnosing and assessing therapeutic efficacy of a mental illness, comprising:
(i) generating, by a processor of a computing device, an image of a virtual multi-dimensional object on a display screen of the computing device for testing a person's susceptibility to a Depth Inversion Illusion (“DII”) having a DII strength level by:
using texture titration to generate a composite image based on a face texture image and the DII strength level,
applying planar texture projection to map the composite image onto the virtual multi-dimensional object to generate a mapped 3-D model, and
generating the image of the virtual multi-dimensional object based on a projected view of the mapped 3-D model from a viewing angle;
(ii) rendering the image of the virtual multi-dimensional object on the display screen;
(iii) collecting first information indicating the person's perceptual response to the DII;
(iv) adjusting the DII strength level to be a second DII strength level and repeating the step of (i) and (ii);
(v) collecting second information indicating the person's perceptual response to the adjusted DII strength level;
(vii) using the first and second information to determine differences between the person's perceptual responses to the DII and reference perception responses of a group of control subjects to the DII; and
(viii) analyzing the differences to determine a severity of the person's mental illness or to assess therapeutic efficacy.

2. The method according to claim 1, wherein the virtual multi-dimensional object is a hollow mask of a face.

3. The method according to claim 1, wherein the first information and the second information each comprises eye movement data captured from one or more sensors that track eye movements of the person in response to the image of the virtual multi-dimensional object on the display screen.

4. The method according to claim 1, wherein the first information and the second information each comprises user-input information specifying the person's answer to at least one question that prompts the person to determine one or more characteristics of the image of the virtual multi-dimensional object on the display screen.

5. The method according to claim 4, wherein the question prompts the person to determine whether the image of the virtual multi-dimensional object on the display screen is perceived as concave or convex.

6. The method according to claim 1, wherein generating the composite image comprises:
generating a composite dot texture image by:
generating a dot texture image comprising a plurality of binary cells each comprising a plurality of pixels, each cell being defined randomly by a value of white or black with equal probability,
generating one or more scaled dot texture images based on the dot texture image, wherein each scale dot texture image is scaled down a percentage from the dot texture image,
aligning the one or more scaled dot texture images with the dot texture image, and
overlaying the one or more aligned dot texture images to the dot texture image to generate the composite dot texture image, wherein each pixel in the composite dot texture image has a value of black if at least one corresponding pixel in the dot texture or the one or more aligned dot texture images has a value of black; otherwise the pixel in the composite dot texture image has a value of white;
aligning the composite dot texture image with the face texture image; and
overlaying a first proportion of the aligned composite dot texture image to a second proportion of the face texture image, wherein the first and second proportions are summed at a value of one.

7. The method according to claim 6, wherein adjusting the DII strength level comprising changing the second proportion for overlaying the aligned composite dot texture image to the face texture image.

8. The method according to claim 1, wherein applying planar texture projection to map the composite image onto virtual multi-dimensional object comprises mapping the composite image onto at least one of concave or convex side of the virtual multi-dimensional object.

9. The method according to claim 1, wherein determining the differences comprises:
determining first data points representing the person's perceptual responses to the DII at a range of DII strength levels;
determining second data points representing perception responses of a group of control subjects to the DII at the range of DII strength levels;
respectively comparing each of the first data points to a corresponding data point in the second data points to determine a difference; and
determine the differences by accumulatively adding the difference for each of the first data points over the range of the DII strength levels.

10. A computing system, comprising:
a processor;
a display screen coupled to the processor; and
a non-transitory computer-readable storage medium comprising programming instructions that are configured to cause the processor to implement a method for diagnosing and assessing therapeutic efficacy of a mental illness, wherein the programming instructions comprise instructions to:
(i) generate an image of a virtual multi-dimensional object on the display screen for testing a person's susceptibility to a Depth Inversion Illusion (“DII”) having a DII strength level by:
using texture titration to generate a composite image based on a face texture image and the DII strength level,
applying planar texture projection to map the composite image onto virtual multi-dimensional object to generate a mapped 3-D model, and
generating the image of the virtual multi-dimensional object based on a view of the mapped 3-D model from a viewing angle;
(ii) render the image of the virtual multi-dimensional object on the display screen;
(iii) collect first information indicating the person's perceptual response to the DII;
(iv) adjust the DII strength level to be a second DII strength level and repeating the steps of (i) and (ii);
(v) collect second information indicating the person's perceptual response to the adjusted DII strength level;
(vi) use the first and second information to determine differences between the person's perceptual responses to the DII and reference perception responses of a group of control subjects to the DII; and
(vii) analyze the differences to determine a severity of the person's mental illness or to assess therapeutic efficacy.

11. The computing system according to claim 10, wherein the virtual multi-dimensional object is a hollow mask of a face.

12. The computing system according to claim 10, further comprising one or more sensors configured to capture eye movement data by tracking eye movements of the person so that the first information and the second information each comprises eye movement data of the person in response to the image of the virtual multi-dimensional object on the display screen.

13. The computing system according to claim 10, wherein the first information and the second information each comprises user-input information specifying a person's answer to at least one question that relates to one or more characteristics of the image of the virtual multi-dimensional object.

14. The computing system according to claim 10, wherein programming instructions for generating the composite image comprise instructions for:
generating a composite dot texture image by:
generating a dot texture image comprising a plurality of binary cells each comprising a plurality of pixels, each cell being defined randomly by a value of white or black with equal probability,
generating one or more scaled dot texture images based on the dot texture image, wherein each scale dot texture image is scaled down a percentage from the dot texture image,
aligning the one or more scaled dot texture images with the dot texture image, and
overlaying the one or more aligned dot texture images to the dot texture image to generate the composite dot texture image, wherein each pixel in the composite dot texture image has a value of black if at least one corresponding pixel in the dot texture or the one or more aligned dot texture images has a value of black; otherwise the pixel in the composite dot texture image has a value of white;
aligning the composite dot texture image with the face texture image; and
overlaying a first proportion of the aligned composite dot texture image to a second proportion of the face texture image, wherein the first and second proportions are summed at a value of one.

15. The computing system according to claim 14, wherein programming instructions for adjusting the DII strength level comprise programming instructions for changing the second proportion for overlaying the aligned composite dot texture image to the face texture image.

16. The computing system according to claim 10, wherein programming instructions for applying planar texture projection to map the composite image onto virtual multi-dimensional object comprise programming instructions for mapping the composite image onto at least one of concave or convex side of the virtual multi-dimensional object.

17. The computing system according to claim 10, wherein programming instructions for determining the differences comprise programming instructions for:
determining first data points representing the person's perceptual responses to the DII at a range of DII strength levels;
determining second data points representing perception responses of a group of control subjects to the DII at the range of DII strength levels;
respectively comparing each of the first data points to a corresponding data point in the second data points to determine a difference; and
determine the differences by accumulatively adding the difference for each of the first data points over the range of the DII strength levels.

18. The computing system according to claim 10, further comprising additional programming instructions configured to:
repeat the step of (i) to create a sequence of images, each containing an image of the virtual multi-dimensional object that corresponds to a viewing angle; and
render the sequence of images in an animation on the display screen.

19. A computing system, comprising:
a processor;
a display screen coupled to the processor; and
a non-transitory computer-readable storage medium comprising programming instructions that are configured to cause the processor to implement a method for diagnosing and assessing therapeutic efficacy of a mental illness, wherein the programming instructions comprise instructions to:
(i) generate an image of a virtual multi-dimensional object on the display screen for testing a person's susceptibility to a Depth Inversion Illusion (“DII”) having a DII strength level by:
using texture titration to generate a composite image based on a face texture image and the DII strength level,
applying planar texture projection to map the composite image onto virtual multi-dimensional object to generate a mapped 3-D model, and
generating the image of the virtual multi-dimensional object based on a view of the mapped 3-D model from a viewing angle; and
(ii) render the image of the virtual multi-dimensional object on the display screen.

20. The computing system according to claim 19, wherein programming instructions for generating the composite image comprise instructions for:
generating a composite dot texture image by:
generating a dot texture image comprising a plurality of binary cells each comprising a plurality of pixels, each cell being defined randomly by a value of white or black with equal probability,
generating one or more scaled dot texture images based on the dot texture image, wherein each scale dot texture image is scaled down a percentage from the dot texture image,
aligning the one or more scaled dot texture images with the dot texture image, and
overlaying the one or more aligned dot texture images to the dot texture image to generate the composite dot texture image, wherein each pixel in the composite dot texture image has a value of black if at least one corresponding pixel in the dot texture or the one or more aligned dot texture images has a value of black; otherwise the pixel in the composite dot texture image has a value of white;
aligning the composite dot texture image with the face texture image; and
overlaying a first proportion of the aligned composite dot texture image to a second proportion of the face texture image, wherein the first and second proportions are summed at a value of one.

21. The computing system of claim 19, further comprising additional programming instructions configured to:
repeat the step of (i) to create a sequence of images, each containing an image of the virtual multi-dimensional object corresponding to a viewing angle; and
render the sequence of images in an animation on the display screen.
